(19) United States
(12) Patent Application Publication
(10) Pub. No.: US 2024/0382734 A1
(43) Pub. Date: Nov. 21, 2024

(54) IMPROVED HEMODIALYSIS PORTS AND METHODS

(71) Applicant: Proviflo, LLC, Clinton, MS (US)

(72) Inventor: Charles David Finch, Jr., Clinton, MS (US)

(73) Assignee: Proviflo, LLC, Clinton, MS (US)

(21) Appl. No.: 18/666,638

(22) Filed: May 16, 2024

Related U.S. Application Data
(60) Provisional application No. 63/467,256, filed on May 17, 2023.

(51) Int. Cl. A61M 39/02; A61M 39/12; A61M 39/26

(52) U.S. Cl. CPC A61M 39/0208; A61M 39/12; A61M 39/26; A61M 2039/0229; A61M 2039/0238; A61M 2205/04; A61M 2205/582

(57) ABSTRACT

Implantable graft-port systems, devices, and methods for establishing access to a fluid-filled internal body space of a patient including the patient's vascular system for blood treatments are described herein. A port device includes a substantially flat surface. The surface is oriented proximal to and substantially parallel with the patient's skin when the device is subcutaneously implanted. The port device includes a tapered seat disposed in a center of the surface and configured to receive a tip of an access tube. The tapered seat is configured to receive the tip of the access tube therethrough. A lid is configured to couple to the substantially flat surface of the port device and includes a guide configured to engage the tip of the access tube and to facilitate percutaneous introduction of the access tube toward the tapered seat irrespective of an introduction angle. The guide includes a continuous sequence of fluted features.

### BACKGROUND OF THE INVENTION

This application claims the benefit of U.S. Provisional Application No. 63/467,256 filed May 17, 2023, the full disclosure of which is incorporated herein by reference in its entirety for all purposes.

### BACKGROUND OF THE INVENTION

The present invention relates generally to the design and use of medical devices, and more particularly to the design and use of implantable graft-port systems, devices, and methods for establishing access to a fluid-filled internal body space of a patient including the patient's vascular system for blood treatments. In general, these blood treatments include, but are not limited to, hemodialysis, hemofiltration, hemodiafiltration, plasmapheresis, ultrafiltration, aquapheresis, n lipid pheresis and hemoperfusion. In the following description, the term “hemodialysis” (or “HD”) is generally used in connection with the present invention, but it is not intended to restrict the use of the device and methods of the present invention to hemodialysis. The subject invention may be used for other blood treatments, drug infusions, or any procedures that require access to a fluid-filled internal body space of a patient, for example. Lock solutions to prevent fowling and infection are also disclosed.

Access to a patient's vascular system can be established by a variety of temporary and permanent devices implanted under the patient's skin. Temporary access can be provided by the direct percutaneous introduction of a needle through the patient's integument and into a blood vessel. While such a direct approach is relatively simple and suitable for some applications, such as intravenous feeding, intravenous drug delivery, and other applications which are limited in time, they are not suitable for hemodialysis, chemotherapy, and extracorporeal procedures that must be repeated periodically, sometimes for the duration of the patient's life. Hemodialysis and hemofiltration (also referred to as hemofiltration), both rely on separate draw and return catheters implanted in a vein and/or artery to allow extra corporeal treatment of the blood. Peritoneal dialysis, in contrast, relies on a single catheter implanted in the peritoneum to permit introduction and withdrawal of dialysate to permit in situ dialysis.

In 2012, the number of patients treated for end stage renal disease (ESRD) was estimated to be about 3 million. Of these patients, about 2.8 million were undergoing dialysis treatment. Medicare spends about $8 billion in dialysis procedures annually. Despite the significant costs, about 20% of dialysis patients in the United States die each year, most often from heart disease or infections. Dialysis treatment of individuals suffering from renal failure requires that blood be withdrawn and cycled through a dialysis machine that performs the function of the failed kidneys. Hemodialysis must be repeated at regular intervals and thus requires repeated punctures using dialysis needles. These relatively large gauge needles are required to promote the high flow rates required during dialysis. Frequent puncturing of autogenous arteriovenous access as well as prosthetic arteriovenous access with large bore needles can cause trauma, conduit degeneration, hematoma formation, pseudoaneurysm formation, loss of patency, or even hemorrhage and exsanguination.

### Implanted Ports:

For hemodialysis and other extracorporeal treatment regimens, a variety of implantable ports have been proposed throughout recent decades. Typically, the port includes a chamber and an access region, such as a septum, where the chamber is attached to an implanted catheter which, in turn, is secured to a blood vessel. In the case of veins, the catheter is typically indwelling and in the case of arteries, the catheter may be attached by conventional anastomosis. These access methods permit only limited flow rates which can be problematic since they prolong the duration of the treatment (i.e., hemodialysis, hemofiltration, plasmaphoresis, apheresis). Moreover, limited flow rates may cause catheter blockages or plugging resulting from fibrin sheath or thrombosis formation over the distal end of the catheter.

Vasca and Biolink were familiar companies founded in the mid-1990s that lead the development of totally implantable HD ports based on the notion that prior implanted ports demonstrated low infection and thrombosis as compared to catheters used for chemotherapy applications. These companies surmised that the experiences learned from chemotherapy catheters could be replicated with HD ports. However, infection was an early problem, and thrombosis complications were only marginally better when compared with HD catheter infection rates. Unexpected problems occurred relating to large needle size, increased frequency of needle puncture and the necessary high blood flow rates, which imposed harsher conditions for hemodialysis access than those encountered during chemotherapy. Vasca and Biolink left the business by 2006 after considerable effort.

Vasca's port was named LifeSite® (FIG. 1). Vasca obtained U.S. approval for commercial distribution of LifeSite® and sold the product for approximately 5 years between about 2000 and 2005. LifeSite® was found to induce complications in patients, including surgical site infections and needle puncture site infections, which were aggravated by poor tissue healing around the needle puncture site. Use of the buttonhole (or “BH”) needle guidance technique in conjunction with LifeSite® was a factor in several infection episodes, in spite of various prophylactic measures (Ross, John J., et al., “Infections Associated with Use of the LifeSite® Hemodialysis Access System” Clinical Infectious Diseases 2002, 35:93-95). Misalignment of the buttonhole-type needle tract with the port's entrance resulted in failures to access correctly. Several factors may have contributed to poor performance, including:
(a) large needle size (i.e., 14 gauge) and poor closure of the needle tract after needle withdrawal;
(b) poor subcutaneous tissue healing and infection of the tissue around the needle tract;
(c) length of the needle tract in subcutaneous tissue, with the perpendicular, protruding needle exiting from patient's skin, susceptible to inadvertent bumping and/or tearing of tissue and dislodgement;
(d) the size and orientation of the implanted LifeSite® created high tensile stress in the tissue acting on the BH tract, which tended to open the BH tract;
(e) sealing/locking of the docked needle within the port was not reliable, and could be compromised by forces acting on the protruding needle, resulting in blood leakage during the HD treatment;
(f) in vivo shifting of the port relative to the BH tract caused misalignment of the port relative to the BH, so that needles were guided away from the port entrance, creating difficulty in accessing the blood and/or causing missed dialysis sessions;
(g) antimicrobial prophylaxis was not “locked” within the luminal passages of the port during the quiescent period, so microbes entering the catheter would not be exposed to a biocide; and
(h) the LifeSite® design was subject to “single fault” failure caused by needle dislodgement.

Biolink's port product was called Dialock® (FIG. 2). Dialock® was evaluated in a pilot clinical trial starting in 1996 and infections occurred quickly. It was realized that tissue infections from needle punctures could be reduced by injecting an antimicrobial “lock” solution into the tissue encapsulating the implanted port. However, serious tissue healing complications, caused by frequent puncturing with large needles, and the difficulty of establishing blood access remained a problem. These and other deficiencies in Dialock® performance would ultimately limit adoption of this port. Biolink Corporation declared bankruptcy around 2004.

One problem with the Dialock® device is that the entry ports are usually inclined at a substantial angle relative to the skin surface through which the access tube (i.e., needle or trocar) is introduced. Such angled access requires that the health care professional introducing the access tube guess the angle and estimate the optimum insertion point on the patient's skin. This uncertainty in the device penetration is perhaps why this type of design necessitates the use of an enlarged “funnel” for receiving and aligning the access tube as it is introduced. It would thus be advantageous to provide access ports having entry passages which are disposed generally “vertically” (i.e., at an angle which is substantially normal to the skin surface through which the access tube is being introduced). By penetrating the access tube “straight in”, it is much easier to align the access tube with the target opening. In this manner, the size of the orifice area can be reduced and the potential for skin damage can be minimized.

Implantable ports typically include a needle-penetrable septum which permits the percutaneous penetration of a needle or trocar into the internal chamber. The chamber, in turn, is connected to one end of the catheter, and the other end of the catheter is indwelling in the blood vessel. While workable, such designs suffer from a number of problems. Repeated penetration of the septum often leads to degradation over time, presenting a substantial risk of small particulates entering the blood stream. The implanted port may also require periodic replacement. Second, the passage of blood through the chamber or plenum will often encounter regions of turbulence or low flow, either of which can degrade the quality of blood over time and add to the time it takes to complete the patient's treatment regime.

Historically, attempts to solve these problems have included internal valve structures which isolate the interior of the port from the lumen of the implanted catheter when the port is not in use. Such valve-enabled ports, however, have their own shortcomings. For example, self-penetrating needles often cannot be used since they will be damaged by and/or cause damage to the port. In such instances, it is frequently necessary to use a catheter combined with a removable stylet, which is both more costly and more inconvenient than use of a simple needle. Moreover, many valved ports have no means or mechanism to assure that the valve is fully opened, particularly when insertion of the access needle opens the valve. Partial insertion of the needle can result in partial opening of the valve which can include a series of deleterious events.

A number of specific valve types have been incorporated into access port designs, including articulating valves such as leaflet valves, ball valves, and flapper valves. All such structures generally require that the access device be passed through the valve itself (i.e., the portion which closes the blood flow path through the valve) in order to cause the valve to open. Such a requirement presents the risk that the valve will be degraded by direct contact with the access device after repeated uses so that portions of the valve may be degraded and released into circulation. Such valves also represent significant risk of failure after repeated use or contact with a sharpened needle. Additionally, such valve structures can damage the access device as it is being introduced there through, thus potentially disrupting valve flow.

Many types of needle-actuated valved ports have been described over the years. Some ports include a duckbill valve which is opened by an elastomeric plug which is elongated by insertion of a needle. So long as the needle is fully inserted, the valve will be fully opened. It would be possible, however, to insert the needle only partially, resulting in only partial opening of the duckbill valve. Such partial opening could significantly degrade and alter the valve performance. Other needle-activated ports include locking mechanisms such as pinch clamps, displaceable balls and other elaborate features that increase the overall size of the port device. Large, bulky implanted ports can be obtrusive and uncomfortable for the patient when implanted. Furthermore, the geometry of some ports, particularly at the needle insertion point, may stretch the skin of the patient increasing the possibility of tearing and subsequent infection.

For these reasons, it would be desirable to provide improved valved implantable access ports for percutaneously accessing a patient's blood vessels, including both arteries and veins. The access ports will comprise a valve structure for isolating the port from an associated implanted catheter when the port is not in use. The valve will preferably provide little or no structure within the blood flow lumen of the access port and will even more preferably not require passage of an access tube, trocar, or the like through the seating portion of a valve in order to open the valve. Furthermore, the port structure including the valve elements therein will have a substantially uniform cross-sectional area and will present no significant constrictions or enlargements to disturb or impede fluid flow there through. The port designs will permit percutaneous access using a conventional needle (i.e., fistula needle or standard trocar) or a proprietary needle without damaging the port or the needle. Still more preferably, the ports will include means for keeping the valve structures open in response to insertion of the needle or other access device without the needle becoming dislodged before the treatment is concluded. It would also be advantageous to provide increased flow rates without increasing the diameter of the catheter to reduce treatment time and thus improve the quality of life for the patient. Ports and valves according to the present invention will meet at least some of these objectives.

Information related to attempts to address these problems can be found in U.S. Pat. Nos. 3,998,222; 4,108,173; 4,181,132; 4,496,343; 4,534,759; 4,569,675; 4,778,452; 4,983,162; 5,053,013; 5,057,084; 5,120,313; 5,180,365; 5,226,879; 5,281,199; 5,263,930; 5,350,360; 5,417,656; 5,421,814; 5,476,451; 5,503,630; 5,520,643; 5,527,277; 5,527,278; 5,562,617; 5,637,088; 5,702,363; 5,704,915; 5,741,228; 5,755,780; 5,954,691; 5,989,239; 6,007,516; 6,022,335; 6,261,257; 6,582,409; 7,056,316; 7,131,192; 7,473,240; 7,803,143; 7,806,122; 8,151,801; 8,348,909 and U.S. Patent Application Publication Numbers 2007/0265584; 2011/0264104; 2014/0018721; 2014/0024998; 2014/0128792 as well as European Patent Application Numbers: EP 1550479; EP 2686033; EP 2300071 and International Patent Application Numbers: WO 95/19200; WO 96/31246; WO 97/047338; WO 98/35710; WO 99/38438; WO 07/061787; WO 09/152488; WO 10/015001; and WO 12/125927, for example.

### Lock Solutions:

The need to leave catheters implanted for a prolonged period raises a number of concerns. First, the catheters can become infected requiring treatment of the patient and often requires removal of the catheter. This is a particular problem with transcutaneous catheters where the skin penetration is a common route of infection. Urinary catheterization exposes patients to increased risk of urinary, kidney and blood (sepsis) infections. Some other catheter-related infectious complications include septic shock, endocarditis, septic arthritis, osteomyelitis, and epidural abscess. Biofilms of infectious bacteria and yeasts often colonize indwelling catheters. Second, implanted catheters can often become plugged or fouled over time. Catheter malfunction is often due to extrinsic and/or intrinsic thrombosis and has been found to be the most common indication for catheter removal. This is a particular problem with intravascular catheters where clotting and intrinsic thrombus formation (i.e., within the catheter lumen) can be problematic. Extrinsic thrombosis, including central venous thrombosis, is also an important and common complication.

To reduce problems associated with thrombus formation, it is now common to “lock” intravascular access catheters between successive uses. Locking typically involves first flushing the catheter with saline to remove blood, medications, cellular debris, and other substances from the catheter lumen. After the catheter has been flushed, a locking solution, typically heparin, is then injected to displace the saline and fill the lumen. The heparin locking solution both excludes blood from the lumen and actively inhibits clotting and thrombus formation within the lumen. To address infection, various antimicrobial substances have been combined with the locking solution in order to inhibit infection at the same time that thrombosis is being inhibited. However, problems with current and continuously emerging resistance to antimicrobial substances, as well as the over-use (and hence the increased risk of developing resistance) of anti-microbials, is an ever-growing concern.

While generally effective, the use of heparin locks suffers from a number of problems and disadvantages. For example, some thrombi may still form at the distal tip of the catheter despite the use of heparin. The need to prepare a heparin solution at the end of every catheter treatment session is time-consuming and presents an opportunity for error by a caregiver. Additionally, heparin has been shown to stimulate biofilm formation which makes it necessary to combine an antimicrobial compound in the heparin lock solution. Heparin is also associated with potentially adverse effects, including heparin-induced thrombocytopenia and bleeding risks.

Various acids have been proposed for use as antimicrobial catheter lock solutions. However, high concentrations of these acids have been shown to cause hemolysis of red blood cells and other harmful effects. Citrate, an ionic form of citric acid, will chelate the divalent cations including the calcium ions in blood and tissue. Serious symptoms have been reported when the ionized calcium blood level decreases. Spillage of extra locking solution into the patient includes miscalculating the lock volume, multiple instillations of solution into the same lumen and even deliberate over injection of solution to clear an occluded catheter. Thus, there is legitimate concern that risks of using concentrated sodium citrate for a catheter lock are not well understood.

Therefore, it would also be desirable to provide improved catheter lock solutions and locking methods to inhibit fouling of the catheter lumen and/or reduce the chance of infection, preferably both. In particular, such methods should be cidal against a broad spectrum of microorganisms and discourage the development of resistant microbes without damaging blood and/or tissue cells. The lock should be relatively inexpensive, non-toxic, easy to store, compatible with the catheter and port materials, safe if inadvertently infused systemically, easy to implement, require minimum or no preparation, and be useful with most or all types of implanted catheters, including hemodialysis and hemofiltration catheters, IV catheters, peritoneal dialysis catheters, urinary catheters, chemotherapy catheters, and the like. At least some of these objectives will be met by the invention described hereinafter.

Information related to attempts to address these problems can be found in U.S. Pat. Nos. 4,114,325; 4,929,242; 5,077,281; 6,635,243; 6,423,706; 6,679,870; 6,685,694; 6,824,532; 6,958,049 and U.S. Patent Application Publication Numbers: 2003/0175323; 2005/0037048; 2005/0043673; 2005/0181008; 2006/0024360; 2006/0052757; 2006/0062850; 2006/0094690; 2006/0177477; 2006/0253063; 2006/0257390; 2007/0292355; 2008/0118544; 2010/0249747 and 2011/0311602 as well as International Patent Application Number: WO 2000/01391, for example. Citrate has been discussed as a locking solution in various concentrations or in combination with other compounds in numerous publications including, for example, Ash et al., ASAIO Journal, (2000) 46 (2): 222; Mandolfo et al., Journal of Vascular Access, (2006) 7 (3): 99-102; Dogra et al., Journal of the American Society of Nephrology, (2002) 13 (8): 2133-2139; and Meeus et al., Blood Purification (2005) 23 (2): 101-105.

Various graft-port devices, methods for establishing access to a vascular system, and lock solutions, including some embodiments of the invention, can mitigate or reduce the effect of, or even take advantage of, some or all of these potential problems.

For the foregoing reasons, there is a legitimate need for effective and efficient ways to provide subcutaneously implantable graft-port systems, devices, and methods for establishing access to a vascular system of a patient that requires periodic ongoing extracorporeal blood treatment.

It would be desirable to leverage the advantages of a graft access while maintaining an easy-to-use port interface in order to decrease miscannulation and promote intra-session hemostasis. It would be particularly beneficial to also provide a graft-port systems, devices and methods for establishing vascular access to a patient to facilitate some or all of the following: 1) reduce the overall size of the implanted port; 2) simplify the locking mechanism to reduce the form factor of the port; 3) reduce the risk of foreign contaminants from invading the port; 4) decrease the incidence of bacteremia and sepsis; 5) simplify the surgical implantation and use of the device for health care professionals; 6) reduce the cross-sectional needle sealing area; 7) enhance overall safety via secure connections; 8) increase the quality of life and reduce treatment pain for the patient; 9) increase blood flow through the device during use; and 10) provide a lock solution to prevent fowling and infection. These attributes would increase treatment efficiency and improve the longevity and quality of life for the patient.

### BRIEF SUMMARY OF THE INVENTION

Certain embodiments of the invention relate to the design and use of implantable graft-port systems, devices, and methods for establishing access to a fluid-filled internal body space of a patient including accessing the patient's vascular system to receive blood treatments. More specifically, some embodiments of the present invention satisfy the need of providing graft-port systems, devices and methods for establishing vascular access while reducing the overall size of the implanted port, simplifying the locking mechanism to reduce the form factor of the port, reducing the risk of foreign contaminants from invading the port, decreasing the incidence of bacteremia and sepsis, simplifying the surgical implantation and use of the device for health care professionals, reducing the cross-sectional needle sealing area, enhancing overall safety via secure connections, increasing the quality of life and reducing treatment pain for the patient, increasing blood flow through the device during use.

According to at least some embodiments, a subcutaneously implanted graft-port device used to establish access to a blood vessel of a patient includes a housing having an inlet opening, an outlet opening, and an interior conduit defined therebetween. The interior conduit is configured to accept a vascular blood flow and the housing includes a substantially flat surface oriented proximal to and substantially parallel with a skin of the patient when the device is subcutaneously implanted. A tapered seat is disposed in a center of the housing and the tapered seat includes an outer perimeter and an inner perimeter that is smaller than the outer perimeter. The tapered seat is configured to receive an access tube through the outer perimeter at the inlet opening. The device further comprises a valve mechanism disposed in the housing at a first position proximal to the outer perimeter of the tapered seat at the inlet opening. The valve mechanism is configured to seal the interior conduit closed to physiologic pressures while allowing for vascular blood flow in the first position. The valve mechanism is configured to be displaced to a second position via percutaneous insertion of the access tube into the tapered seat to open the valve mechanism and allow continued vascular blood flow through the interior conduit.

In various embodiments, the valve mechanism is configured to be displaced to the second position by application of actuation pressure that does not cause pain to a patient. The valve mechanism is configured to return to the first position and resume sealing of the interior conduit upon removal of the access tube from the tapered seat at a conclusion of a treatment session. The access tube may be a needle. The treatment session may include any of hemodialysis, hemofiltration, hemodiafiltration, plasmapheresis, ultrafiltration, aquapheresis, lipid pheresis, chemotherapy, hemoperfusion, peritoneal dialysis, and pleural drainage. The valve mechanism in the second position allows for continued vascular blood flow through the interior conduit unobstructed by the access tube. The valve mechanism may extend 45 degrees from a longitudinal axis of the housing. In at least some embodiments, the valve mechanism is a movable spherical element.

The device further comprises a sealing element located at the outer perimeter of the tapered seat configured to seal the valve mechanism. The sealing element may be an O-ring for further facilitating sealing of the interior conduit.

The tapered seat comprises a conical surface extending between the outer perimeter and the inner perimeter. For example, the tapered seat may include a taper angle of at least or less than 1 degree from the outer perimeter to the inner perimeter.

The device further comprises a lid configured to couple with the substantially flat surface of the housing. The lid includes a continuous sequence of fluted features. The fluted features include at least one ridge element elevated from a surface, wherein each ridge element terminates adjacent the outer perimeter of the tapered seat. The fluted features are configured and located to guide a tip of the access tube toward the outer perimeter of the tapered seat so as to facilitate percutaneous introduction of the access tube into the tapered seat. The fluted features include at least one groove element depressed from the surface, wherein each groove element terminates adjacent the outer perimeter of the tapered seat, and wherein the at least one groove element alternates with the at least one ridge element.

In some embodiments, the housing further includes a transparent material, one or more light emitting diodes, and a receiver coil for receiving an electrical current. The one or more light emitting diodes are configured to illuminate the tapered seat when an electromagnetic induction chip is placed external to the patient and substantially above the subcutaneously implanted device, the electromagnetic induction chip producing a voltage in the receiver coil, the voltage powering the one or more light emitting diodes so that the tapered seat of the subcutaneously implanted device is visible to a user of the device.

According to various embodiments, at least a portion of the housing is sufficiently nano-porous so as to permit tuned diffusion of an anti-microbial agent outward from the interior conduit of the housing to an outer surface of the housing so that infection is inhibited.

According to at least some other embodiments, an implantable port device for establishing access to a blood vessel of a patient includes a port device. The port device includes a substantially flat surface that is oriented proximal to and substantially parallel with a skin of the patient when the device is subcutaneously implanted. The port device includes a tapered seat disposed in a center of the substantially flat surface and configured to receive a tip of an access tube. The tapered seat includes a proximal portion, a distal portion, and a conical surface extending between the proximal portion and the distal portion. The proximal portion of the tapered seat is configured to receive the tip of the access tube therethrough. The port device includes an interface surface configured to engage (i) the blood vessel of the patient or (ii) a vascular access catheter. The interface surface includes an aperture in fluid communication with the distal portion of the tapered seat. At least a portion of the port device is sufficiently nano-porous so as to permit tuned diffusion of an anti-microbial agent outward from a conduit of the port device to an outer surface of the port device so that infection is inhibited.

In various embodiments, at least a portion of the port device is characterized as having an average pore diameter that is sufficiently porous to allow an antimicrobial solution to permeate the port device and, preferably, pass outwardly (i.e., seep, ooze, leak, diffuse) into the tissue region surrounding the port device. At least a portion of the port device comprises a nano-porous material such as polypropylene or a ceramic, or any combination thereof. The anti-microbial agent comprises sodium hypochlorite, calcium hypochlorite, sodium oxychlorosone, alcohols, aldehydes, halides, providone iodine, peroxides, antibiotics, etc., or any combination thereof. In various embodiments, the lid is non-porous, and the lid comprises a non-porous ceramic or metal.

In at least some embodiments, the tapered seat comprises a taper angle of at least or less than 1 degree from the proximal portion to the distal portion. The conduit may extend between the proximal portion of the tapered seat and the interface surface.

According to at least some embodiments, the port device further comprises a valve mechanism disposed in the port device at a first position within the proximal portion of the tapered seat. The valve mechanism is configured to seal the conduit closed to physiologic pressures while allowing for vascular blood flow in the first position. The valve mechanism is configured to be displaced to a second position via percutaneous insertion of the access tube into the tapered seat to open the valve mechanism and allow continued vascular blood flow through the conduit.

The device further comprises a lid configured to couple with the substantially flat surface of the housing. The lid includes a continuous sequence of fluted features. The fluted features include at least one ridge element elevated from a surface, wherein each ridge element terminates adjacent the outer perimeter of the tapered seat. The fluted features are configured and located to guide a tip of the access tube toward the outer perimeter of the tapered seat so as to facilitate percutaneous introduction of the access tube into the tapered seat. The fluted features include at least one groove element depressed from the surface, wherein each groove element terminates adjacent the outer perimeter of the tapered seat, and wherein the at least one groove element alternates with the at least one ridge element.

In some embodiments, the port device further includes a transparent material, one or more light emitting diodes, and a receiver coil for receiving an electrical current. The one or more light emitting diodes are configured to illuminate the tapered seat when an electromagnetic induction chip is placed external to the patient and substantially above the subcutaneously implanted device, the electromagnetic induction chip producing a voltage in the receiver coil, the voltage powering the one or more light emitting diodes so that the tapered seat of the subcutaneously implanted device is visible to a user of the device.

According to at least some other embodiments, a two-part implantable port system for establishing access to a blood vessel of a patient includes a port device. The port device includes a substantially flat surface. The substantially flat surface oriented proximal to and substantially parallel with a skin of the patient when the device is subcutaneously implanted. The port device further includes a tapered seat disposed in a center of the substantially flat surface and configured to receive a tip of an access tube. The tapered seat includes a proximal portion, a distal portion, and a first conical surface extending between the proximal portion and the distal portion. The proximal portion of the tapered seat is configured to receive the tip of the access tube therethrough. The port device further includes an interface surface configured to engage (i) the blood vessel of the patient or (ii) a vascular access catheter, the interface surface having an aperture in fluid communication with the distal portion of the tapered seat. The system further includes a lid configured to couple to the substantially flat surface of the port device and the lid includes a guide configured to engage the tip of the access tube and to facilitate percutaneous introduction of the access tube toward the tapered seat irrespective of an introduction angle and the guide includes a continuous sequence of fluted features.

In various embodiments, the fluted features include at least one ridge element elevated from a top surface, wherein each ridge element terminates adjacent the outer perimeter of the tapered seat. The fluted features are configured and located to guide a tip of the access tube toward the outer perimeter of the tapered seat so as to facilitate percutaneous introduction of the access tube into the tapered seat. The fluted features include at least one groove element depressed from the top surface, wherein each groove element terminates adjacent the outer perimeter of the tapered seat, and wherein the at least one groove element alternates with the at least one ridge element. The top surface may be substantially flat, or the top surface may include a second conical surface having an angle within a range from 10 degrees and 25 degrees, inclusive.

According to various embodiments, at least a portion of the port device is sufficiently nano-porous so as to permit tuned diffusion of an anti-microbial agent outward from the interior conduit of the port device to an outer surface of the port device so that infection is inhibited.

In some embodiments, the port device further includes a transparent material, one or more light emitting diodes, and a receiver coil for receiving an electrical current. The one or more light emitting diodes are configured to illuminate the tapered seat when an electromagnetic induction chip is placed external to the patient and substantially above the subcutaneously implanted device, the electromagnetic induction chip producing a voltage in the receiver coil, the voltage powering the one or more light emitting diodes so that the tapered seat of the subcutaneously implanted device is visible to a user of the device.

These and other features, aspects, and advantages of various embodiments of the invention will become better understood with regard to the following description, appended claims, accompanying drawings, and abstract.

### BRIEF DESCRIPTION OF THE DRAWINGS

FIG. 1 illustrates a conventional implantable hemodialysis port.

FIG. 2 illustrates another conventional implantable hemodialysis port.

FIG. 3A is a top perspective view of the port device according to an embodiment of the present invention.

FIG. 3B is a top view of the port device of FIG. 3A according to an embodiment of the present invention.

FIG. 3C is a front view of the port device according to an embodiment of the present invention.

FIG. 3D is a cross-sectional bottom view of the port device of FIG. 3A according to an embodiment of the present invention.

FIG. 3E is a side view of the port device of FIG. 3A according to an embodiment of the present invention.

FIG. 3F is a partial cross-sectional side view of the port device of FIG. 3A according to an embodiment of the present invention.

FIG. 3G is an enlarged view of the partial cross-sectional side view of FIG. 3F.

FIG. 3H is a partial cross-sectional view facing the rear of the port device of FIG. 3A according to an embodiment of the present invention.

FIG. 4A is a top perspective view of a lid according to an embodiment of the present invention.

FIG. 4B is a top view of the lid of FIG. 4A according to an embodiment of the present invention.

FIG. 4C is a side view of the lid of FIG. 4A according to an embodiment of the present invention.

FIG. 4D is a cross-sectional side view of the lid of FIG. 4A according to an embodiment of the present invention.

FIG. 4E is a bottom view of the lid of FIG. 4A according to an embodiment of the present invention.

FIG. 5A is a cross-sectional view of a valve mechanism in a first position according to an embodiment of the present invention.

FIG. 5B is a cross-sectional view of a valve mechanism in a second position according to an embodiment of the present invention.

FIG. 6 is an exploded view of a two-part implantable graft system according to an embodiment of the present invention.

FIG. 7 is a view of an assembly including a two-part implantable graft system according to an embodiment of the present invention.

FIG. 8 is a flowchart of a method according to an embodiment of the present invention.

FIG. 9 illustrates an exemplary connector device for a catheter of an implantable graft system according to an embodiment of the present invention.

FIG. 10 is a view of the connector device of FIG. 9 in an open position and a closed position according to an embodiment of the present invention.

FIG. 11 is a cross-sectional view of the connector device of FIG. 9 according to an embodiment of the present invention.

### DETAILED DESCRIPTION OF THE INVENTION

Various embodiments of the invention relate to the design and use of implantable graft-port systems, devices, and methods for establishing access to a fluid-filled internal body space of a patient including accessing the patient's vascular system to receive blood treatments.

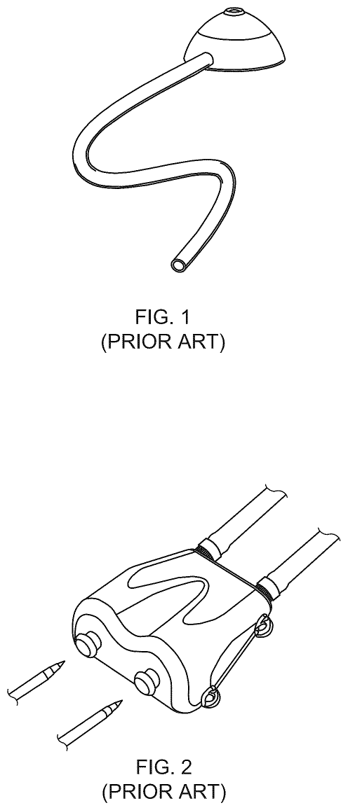

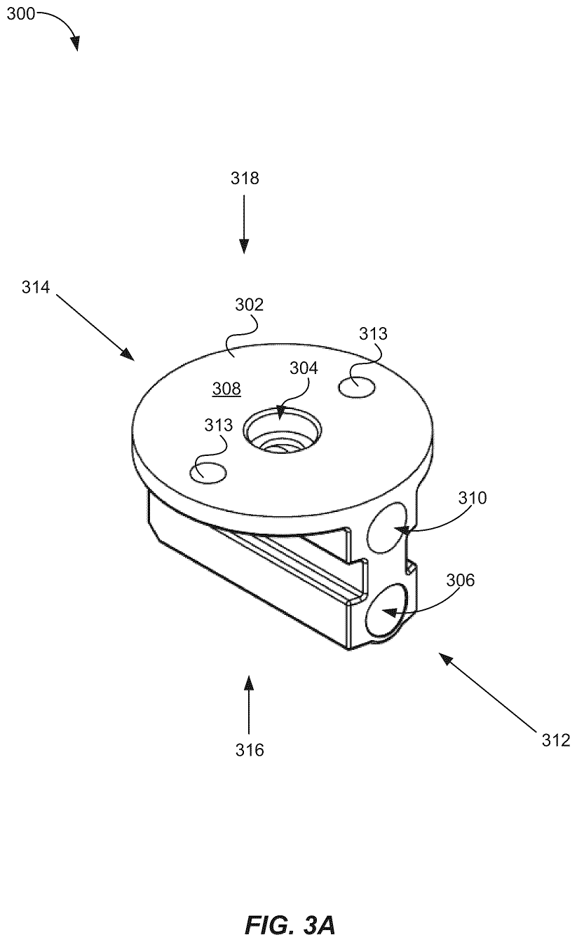

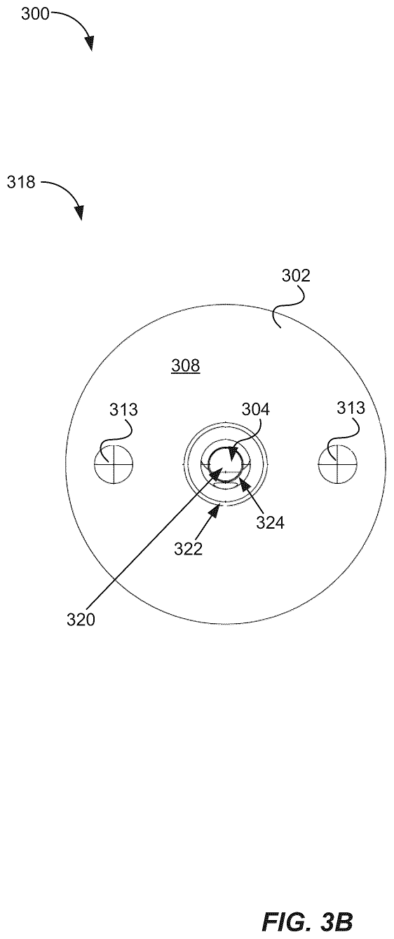

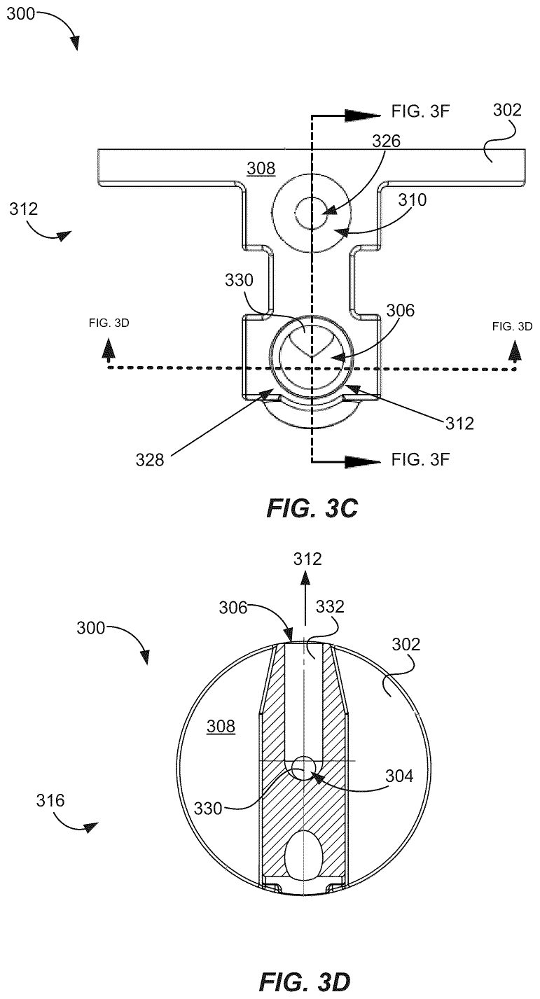

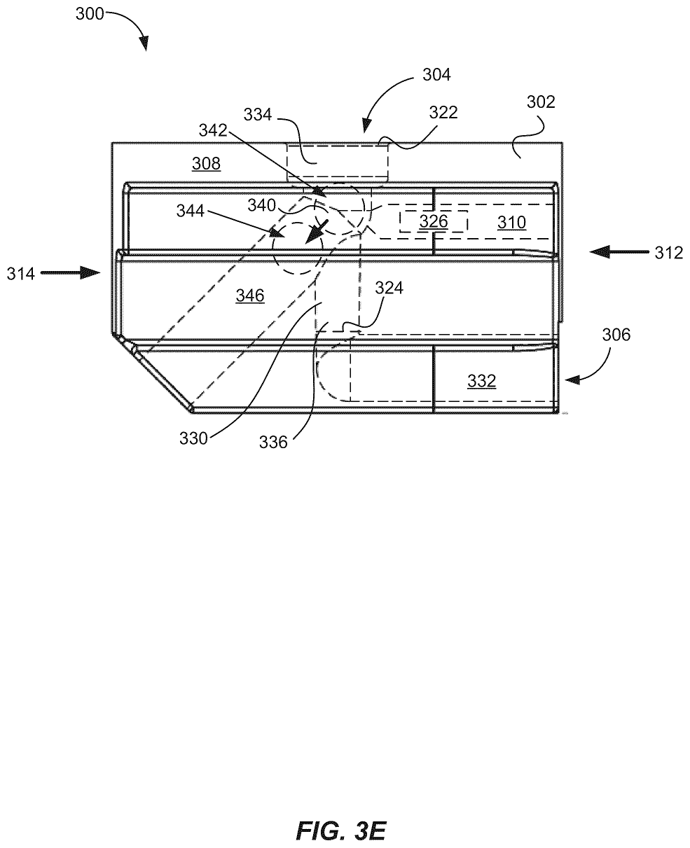

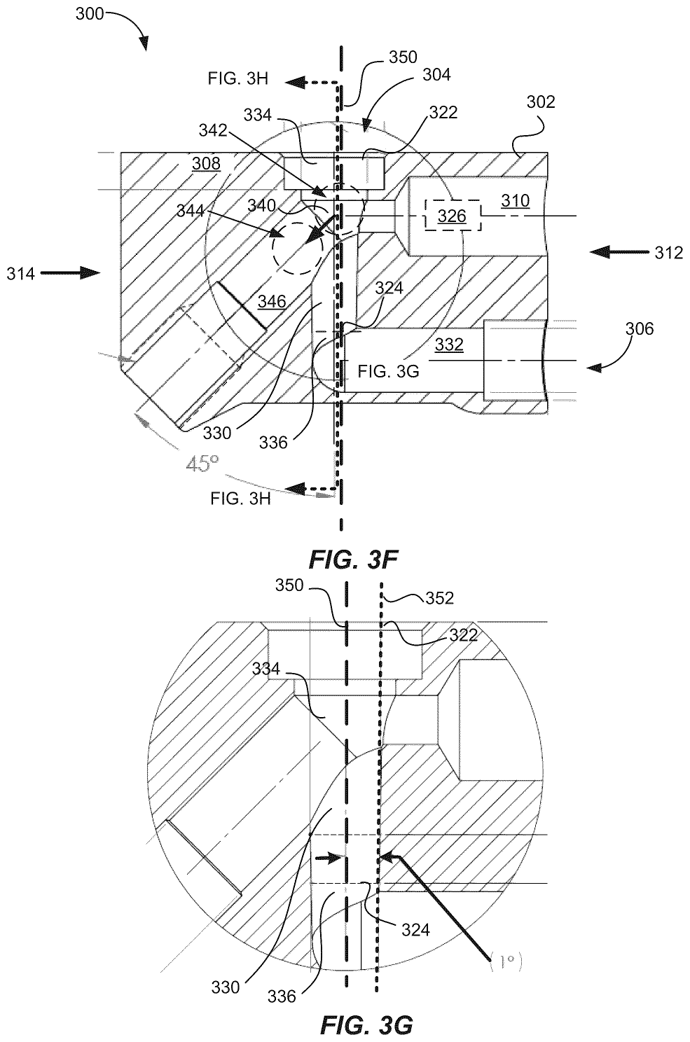

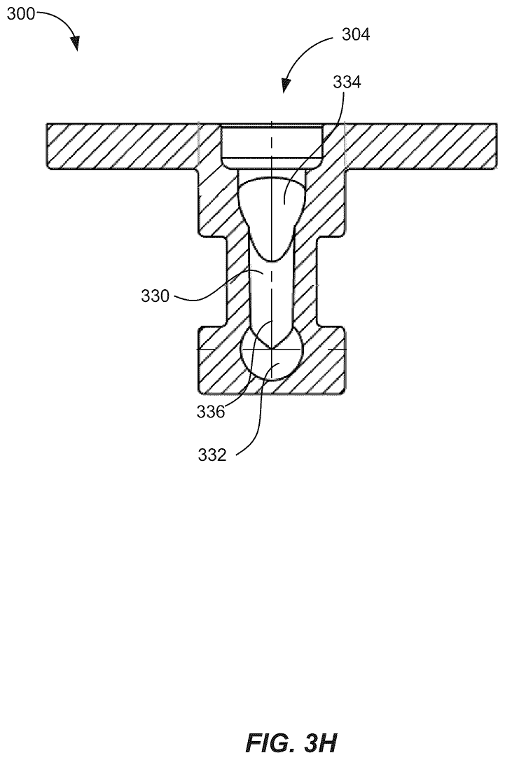

FIGS. 3A-3H describe components of a port device from various perspectives and the same or similar numbering should be assumed to have the same or similar meaning throughout the present disclosure unless otherwise noted herein. FIG. 3A is a top perspective view of the port device according to an embodiment of the present invention. Port device 300 is a subcutaneously implanted graft-port device used to establish access to a blood vessel of a patient. A patient having a port device 300 implanted typically requires repeated vascular access over a period of time, as detailed above. For example, vascular access may be established for performing a treatment such as hemodialysis, hemofiltration, hemodiafiltration, plasmapheresis, ultrafiltration, aquapheresis, lipid pheresis, chemotherapy, hemoperfusion, peritoneal dialysis, or pleural drainage. The port device 300 includes a housing 302 having an inlet opening 304 and an outlet opening 306. The housing 302 of the port device 300 includes a substantially flat, top surface 308 that is oriented proximal to and substantially parallel with a skin of patient when the port device 300 is subcutaneously implanted. Unlike the domed geometry of some prior art devices such as FIG. 1, the flat (plateau-like) surface eases skin tension to prevent stretching and/or tearing of the skin. Insertion of the access tube into the tapered seat opens an interior conduit of the housing 302 to physiological pressure and continuous blood flow through the interior conduit. In some embodiments, and as shown in FIG. 3A, the substantially flat, top surface 308 includes a smooth, non-angled surface. In other embodiments, the substantially flat, top surface 308 may include a texture or pattern (e.g., a repeated sequence of surface features) which facilitates adhesion of the housing 302 to a lid (not shown and described in further detail below).

In at least some embodiments, the housing 302 of the port device 300 includes a transparent material 310 for illuminating a tapered seat (not shown) of the housing 302 of the port device 300, to be described in further detail below. The transparent material 310 may be included with one or more light emitting diodes (LED lights) and a receiver coil for receiving an electrical current. Placing an electromagnetic induction chip external to the patient and substantially above the subcutaneously implanted device induces a voltage in the receiver coil. The voltage powers the lights to visually reveal a location of the subcutaneously implanted device to a user (e.g., the patient, a health care professional, another user, etc.). In this manner, the lights are visible through the skin to reveal the location of the port device 300 just under the patient's skin.

As shown in FIG. 3A, one or more fastening apertures 313 may extend through the top surface 308 of the housing 302 of the port device 300. Although two fastening apertures 313 are shown, one fastening aperture 313, two fastening apertures 313, three fastening apertures 313, etc., may be used. Fastening apertures 313 may be configured to receive a fastener for fastening the housing 302 to a lid (not shown, to be described in further detail below). The fastener may by any type known in the art including a screw, a bolt, a nail, etc. In other embodiments, the housing 302 and the lid may be fastened using other mechanisms including an adhesive, magnetization, forming the housing 302 and the lid as an integral piece, etc.

In various embodiments, at least a portion of the housing 302 is sufficiently nano-porous so as to permit tuned diffusion of an anti-microbial agent outward from an interior conduit (not shown) of the housing 302 to an outer surface of the housing 302 so that infection is inhibited. For example, at least a portion of the housing 302 is characterized as having an average pore diameter that is sufficiently porous to allow an antimicrobial solution to permeate the port device and, preferably, pass outwardly (i.e., seep, ooze, leak, diffuse) into the tissue region surrounding the port device. At least a portion of the housing 302 comprises a nano-porous material such as polypropylene or a ceramic, or any combination thereof. The anti-microbial agent comprises sodium hypochlorite, calcium hypochlorite, sodium oxychlorosone, alcohols, aldehydes, halides, providone iodine, peroxides, antibiotics, etc., or any combination thereof. The anti-microbial agent may be advantageously renewed with each use (e.g., treatment) of the port device 300 for continued resistance to infection or fowling of the port device 300. In various embodiments, a corresponding lid (to be described in further detail below) is non-porous and the lid comprises a non-porous ceramic or metal.

Various embodiments of the port device 300 are described herein with respect to a front view 312 and a rear view 314 of the port device 300. A bottom view 316 and a top view 318 are also used to describe various embodiments of the port device 300. Dotted lines used throughout FIGS. 3A-3H may indicate relative positions from where cross-sections described with respect to other FIGS. are taken. Some elements may be hidden from view or otherwise removed for clarity in at least some FIGS.

FIG. 3B is a top view of the port device of FIG. 3A according to an embodiment of the present invention. FIG. 3B includes a top view 318 of the housing 302 of the port device 300. The top surface 308 is shown having the inlet opening 304 disposed in a center of the housing 302. The inlet opening 304 leads to the tapered seat 320 disposed in the center of the housing 302. The tapered seat 320 includes an outer perimeter 322 and the tapered seat is configured to receive an access tube (not shown) through the outer perimeter 322 at the inlet opening 304. In various embodiments, the access tube may be a needle, a trocar, a syringe, etc. The tapered seat 320 also includes an inner perimeter 324 that is smaller than the outer perimeter 322.

FIG. 3C is a front view of the port device according to an embodiment of the present invention. FIG. 3C includes a front view 312 of the housing 302 of the port device 300. The front view 312 of the housing 302 illustrates the outlet opening 306. The outlet opening 306 defines an interface surface 328 that is configured to engage a tubular structure, e.g., a blood vessel of the patient, a synthetic tube grafted to the blood vessel, or a vascular access catheter. The interface surface 328 includes an aperture (e.g., the outlet opening 306) that is in fluid communication with a distal portion of the tapered seat 330, to be described in further detail below.

In some embodiments, the interface surface 328 is coupled to a catheter (not shown). The catheter may be attached to the port device 300 and used to access a vessel. The catheter may be implanted under the skin of the patient proximate to the implanted port device 300. The catheter may be implanted under the skin between a target vessel, typically a vein, and the implanted port device 300. The outside walls of the catheter may be proximate to the inside walls of the vessel so that the catheter takes up most or all of the space of the vessel lumen, in a manner which would be appreciated by one having ordinary skill in the art upon reading the present disclosure. In this way, all or the majority of the blood flow path flows through a conduit of the catheter. During hemodialysis, for example, blood may be withdrawn through the catheter, through the port device 300 (e.g., through the interior conduit 332 and the tapered seat 330 of the port device 300), and externally through an inserted access tube to percutaneously access the port device 300. Alternatively, the port device 300 and the catheter could be used to return treated blood to the patient. This configuration provides a more efficient therapy and also reduces blood flow turbulence, clotting, and other hemodynamic consequences.

The port device 300 fits snugly around the external diameter of the native vessel or synthetic tube to compress the vessel and provide extrinsic pressure to maintain hemostasis. The vessel wall tension acts as a tamponade to close or block the wound left from the access tube after the tube is withdrawn. The tamponade promotes natural blood clotting and encourages intrinsic coagulation at the puncture site on the skin where the access tube penetrated. This prevents bleeding and promotes healing. Alternatively, the vessel may be secured in a manner known in the art such that the top surface 308 of the port device 300 is maintained in an orientation essentially parallel with the skin when subcutaneously implanted. Attaching the port device 300 with sutures may also prevent movement, migration, or wobble when the access tube is inserted into the port device 300.

In some embodiments, a catheter interfaces with the blood vessel or other body space filled with (or potentially filled with) a fluid (i.e., blood, urine, cerebral-spinal fluid, etc.). It is contemplated that the port device 300 can be used for various therapies beyond hemodialysis with little or no modification. Some other therapeutic uses may include peritoneal dialysis, urinary tract drainage, pleural effusion, and cerebral-spinal fluid drainage. For example, the port device 300 may be used to administer insulin.

As shown, the front view 312 of the housing 302 includes a transparent material 310 for illuminating a tapered seat (not shown) of the housing 302 of the port device 300, to be described in further detail below. The transparent material 310 may be included with one or more light emitting diodes (LED lights) 326 and a receiver coil (not shown) for receiving an electrical current. The LED lights may include ruby, sapphire, or other durable, radiant material visible through the skin of the patient. Placing an electromagnetic induction chip external to the patient and substantially above the subcutaneously implanted device induces a voltage in the receiver coil. The voltage powers the lights to visually reveal a location of the subcutaneously implanted device to a user (e.g., the patient, a health care professional, another user, etc.). In this manner, the one or more LED lights 326 are visible through the skin to reveal the location of the port device 300 just under the patient's skin.

FIG. 3D is a cross-sectional bottom view of the port device of FIG. 3A according to an embodiment of the present invention. The cross-section of the bottom view 316 of the housing 302 of the port device 300 illustrates the distal portion of the tapered seat 330 and at least a portion of an interior conduit 332 of the housing 302. The interior conduit 332 is defined between the inlet opening 304 (e.g., which would be visible from the bottom view 316 through the tapered seat 330 as shown) and the outlet opening 306. The interior conduit 332 is configured to accept a vascular blood flow. The interior conduit 332 is 90 degrees relative to the outlet opening 306. For example, the port device 300 is used to treat a patient by establishing access to a vascular blood flow via at least a portion of the interior conduit 332 of the housing 302 of the port device.

FIG. 3E is a side view of the port device of FIG. 3A according to an embodiment of the present invention. As shown in FIG. 3E, the tapered seat 330 includes a proximal portion 334 having the outer perimeter 322 and a distal portion 336 having an inner perimeter 324. In various embodiments, the inner perimeter 324 is smaller than the outer perimeter 322. Accordingly, in at least some embodiments, the tapered seat 330 includes a conical surface extending between the outer perimeter 322 and the inner perimeter 324. In other words, the tapered seat 330 including an outer perimeter 322 larger than an inner perimeter 324 includes a taper angle of at least or less than 1 degree from the outer perimeter 322 to the inner perimeter 324. The interior conduit 332 is defined between the inlet opening 304 and the outlet opening 306 and the interior conduit 332 is in fluid communication with the distal portion 336 of the tapered seat 330.

As illustrated in FIG. 3E, a valve mechanism 340 is disposed in the housing 302 at a first position 342 proximal to the outer perimeter 322 of the tapered seat 330 at the inlet opening 304. As shown, the valve mechanism 340 includes a spherical element. The inlet opening 304 and/or the proximal portion 334 of the tapered seat 330 are closed to blood flow when the interior conduit 332 and the distal portion 336 of the tapered seat 330 are occluded by the valve mechanism 340 (e.g., a moveable spherical element (ball)). The spherical element may include synthetic sapphire, stainless steel, or other hard material that is hard and lubricious. When an access tube is inserted through the inlet opening 304, the valve mechanism 340 is laterally displaced by the access tube as the access tube is displaced by the access tube.

The valve mechanism 340 is configured to seal the interior conduit 332 closed to physiologic pressures while allowing for vascular blood flow in the first position 342. The valve mechanism 340 is configured to be displaced to a second position 344 via percutaneous insertion of an access tube into the tapered seat 330 to open the valve mechanism 340 and allow continued vascular blood flow through the interior conduit 332. According to at least some embodiments, the second position 344 includes the valve mechanism 340 being at least partially disposed in a valve conduit 346. The valve conduit 346 extends as part of the valve mechanism 340 that extends 45 degrees from a longitudinal axis of the housing 302. The valve conduit 346 provides a space for the movement of the valve mechanism 340. In some embodiments, valve conduit 346 may include an elastomeric spring or silicone ring (both not shown), to accommodate the displaced valve mechanism 340. Other material that adds a resistant force against a displaced valve mechanism 340 may be used in the valve conduit 346.

The valve mechanism 340 in the second position 344 allows for continued vascular blood flow through the interior conduit 332 unobstructed by the access tube. The valve mechanism 340 is configured to return to the first position and resume sealing of the interior conduit 332 upon removal of the access tube from the tapered seat 330 at a conclusion of a treatment session or upon removal of the access tube from the tapered seat 330 for any other reason.

In various embodiments, the valve mechanism 340 is configured to be displaced from the first position 342 to the second position 344 by application of an actuation pressure in a range that does not result in patient discomfort or substantially reduces patient discomfort compared to conventional needle-actuated valve mechanisms. Advantageously, the design of the valve mechanism 340 described according to embodiments included herein requires significantly less pressure to actuate than traditional valve mechanisms. A patient experiences significantly less discomfort when less pressure is applied to a port device (e.g., when access to vascular blood flow is established). For example, having the valve mechanism 340 more proximate to the inlet opening 304 compared to other designs increases the mechanical advantage at the beginning of the percutaneous insertion of the access tube into the port device 300. Less pressure on the valve mechanism 340 results in less pain to the patient as the valve mechanism 340 is displaced and reduces injury to the tissue pocket supporting the implanted device.

In at least some embodiments, a sealing element (not shown) is provided at the outer perimeter 322 of the tapered seat 330 configured to seal the valve mechanism 340. The sealing element may be an O-ring that is compressed into the tapered seat 330 when the valve mechanism 340 is open (e.g., in the second position 344) to seal the valve mechanism 340 and prevent leakage.

According to at least some embodiments, housing 302 includes a transparent material 310 for illuminating a tapered seat (not shown) of the housing 302 of the port device 300. In some approaches, the transparent material 310 is an empty or substantially empty conduit. The transparent material 310 may be included with one or more light emitting diodes (LED lights) 326 and a receiver coil (not shown) for receiving an electrical current. Placing an electromagnetic induction chip external to the patient and substantially above the subcutaneously implanted device induces a voltage in the receiver coil. The voltage powers the lights to visually reveal a location of the subcutaneously implanted device to a user (e.g., the patient, a health care professional, another user, etc.). In this manner, the one or more LED lights 326 are visible through the skin to reveal the location of the port device 300 just under the patient's skin.

FIG. 3F is a partial cross-sectional side view of the port device of FIG. 3A according to an embodiment of the present invention. FIG. 3F includes the same or similar elements as those described with respect to a longitudinal axis 350 of the housing 302. FIG. 3E. The 45 degree angle from the longitudinal axis 350. An inset for FIG. 3G is shown and a position for the partial cross-section of FIG. 3H.

FIG. 3G is an enlarged view of the partial cross-sectional side view of FIG. 3F. FIG. 3G includes the inset of FIG. 3F and illustrates the taper angle 352 of the tapered seat 330 having a proximal portion 334 and a distal portion 336. The taper of the tapered seat 330 extends at least from the outer perimeter 322 of the tapered seat 330 to the inner perimeter 324 of the tapered seat 330. As shown, the taper angle 352 is 1 degree on each side of the longitudinal axis 350. The taper angle 352, according to various embodiments described herein may be less than or equal to 1 degree from the longitudinal axis 350 including, but not limited to, 0.75 degrees, 0.5 degrees, 0.25 degrees, etc.

FIG. 3H is a partial cross-sectional view facing the rear of the port device of FIG. 3A according to an embodiment of the present invention. FIG. 3H illustrates the transition between the tapered seat 330, specifically, the distal portion 336 of the tapered seat 330, and the interior conduit 332. The interior conduit 332 is configured to accept a vascular blood flow which continues through the tapered seat 330. The vascular blood flow may be sealed when the valve mechanism (such as valve mechanism 340, not shown) is in the first position to seal the inlet opening 304.

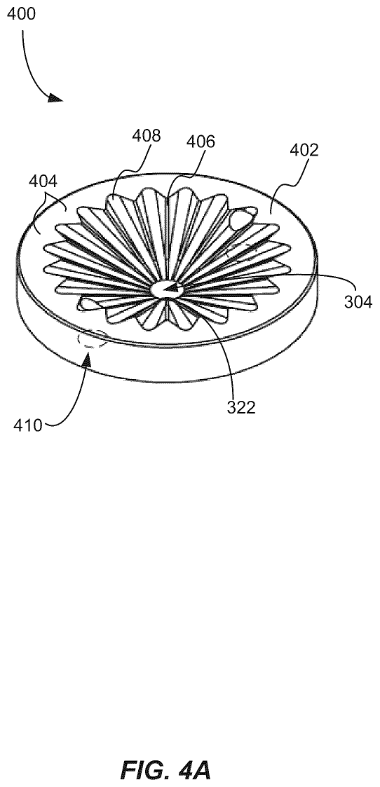

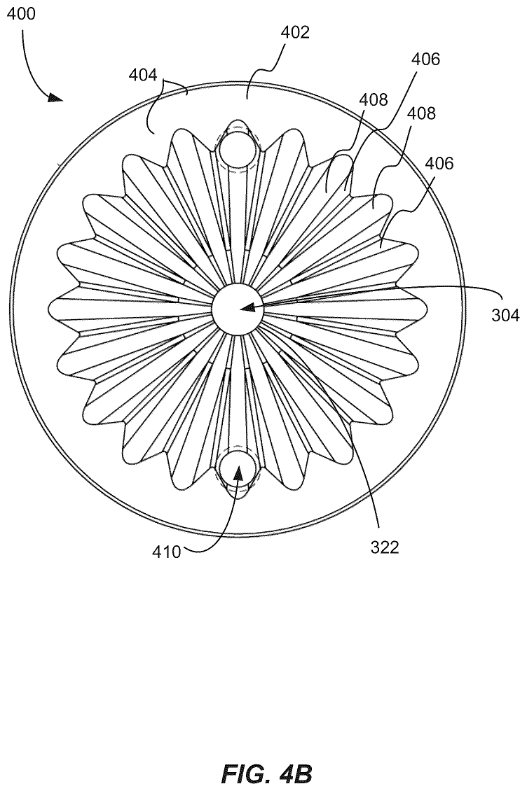

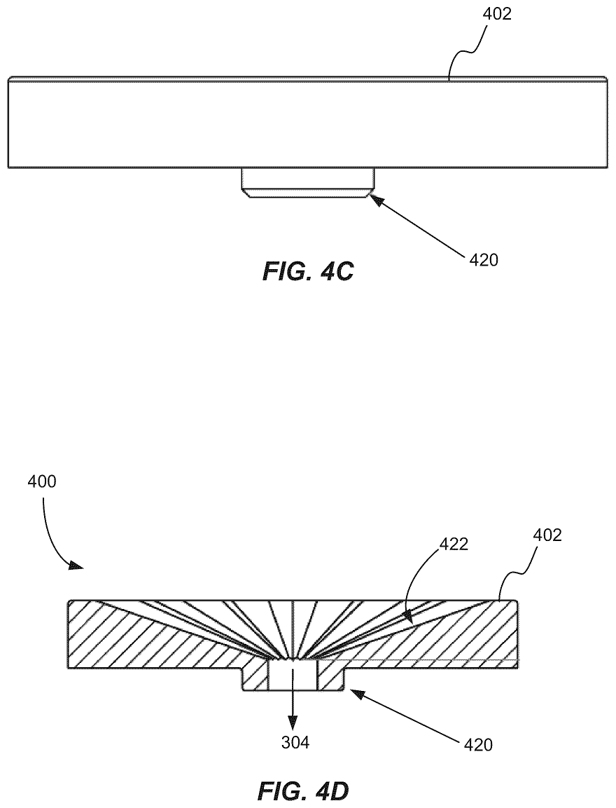

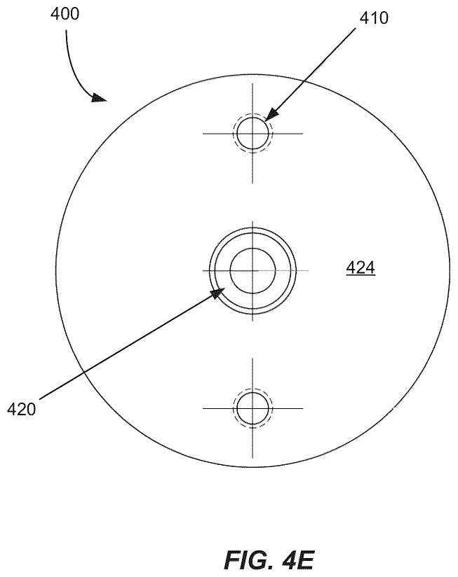

FIG. 4A is a top perspective view of a lid according to an embodiment of the present invention. The same or similar numbering as used in other FIGS. may be used to describe elements shown in FIGS. 4A-4E and should be interpreted to have the same or similar meaning as used elsewhere in the present disclosure. The lid 400, according to various embodiments, in combination with a port device (such as port device 300 described with respect to FIGS. 3A-3H), to form a two-part implantable port system for establishing access to a blood vessel of a patient. In various embodiments, the lid 400 is configured to couple with a substantially flat surface of a port device housing (such top surface 308 of the housing 302 of the port device 300 described with respect to FIGS. 3A-3H). As shown, the lid 400 includes a guide 402 having a continuous sequence of fluted features 404. The guide 402 of the lid 400 is configured to engage the tip of an access tube and to facilitate percutaneous introduction of the access tube toward the tapered seat of the housing (such as tapered seat 330 described with respect to FIGS. 3A-3H) irrespective of an introduction angle. In other words, no matter how a user approaches the two-part implantable port system (including the lid 400 and the port device 300), the access tube is directed to the inlet opening 304 and the tapered seat by the guide 402. For example, the access tube punctures the skin in the vicinity of the inlet opening 304 of the tapered seat. The tip of the access tube engages (e.g., bumps into) elements of the guide 402 and the access tube is moved along the guide 402 until if finds the inlet opening 304.

In some embodiments, the lid 400 can be palpated when it is implanted just under the skin. While it is possible to pierce the skin with the access tube and directly insert it into the tapered seat, a more likely scenario is to palpate the subcutaneously implanted lid 400 and feel the circular edges of the flat surface (which is about the diameter of a dime). The diameter may be about 14 mm but is scalable for alternative applications. In addition, or alternatively, LED lights, such as those described above, may be used to locate the implanted system. For example, the LED lights may be used to validate the position and/or direction of the two-part system.

Fluted features 404 include at least one ridge element 406 elevated from a top surface (e.g., of the guide 402). Each ridge element 406 may be rounded or flat on top. Each ridge element 406 terminates adjacent an outer perimeter 322 of the tapered seat. Fluted features 404 include at least one groove element 408 depressed from a top surface of the guide 402. Each groove element 408 may be rounded or flat at the bottom. Each groove element 408 terminates adjacent the outer perimeter 322 of the tapered seat. According to various embodiments, at least one groove element 408 alternates with at least one ridge element 406. The ridge elements 406 and the groove elements 408 may be arranged in any pattern with each pattern terminating adjacent the outer perimeter 322 of the tapered seat. For example, the fluted features 404 are arranged in a target pattern, as shown, with the outer perimeter of the tapered seat located nearest the center of the target pattern. The pattern may include a combination of distances between the elements (e.g., ridge elements 406 and groove elements 408).

The top surface of the guide 402 may be substantially flat in some embodiments. In other embodiments, the top surface of the guide 402 includes a second conical surface having an angle within a range from 10 degrees and 25 degrees, inclusive, as shown. For example, the fluted features 404 are directed to a center and downward from an otherwise level surface of the guide.

In various embodiments, the lid 400 is a non-porous material including a non-porous ceramic or a metal such as titanium. In some embodiments, the lid 400 is a single molded device produced using an injection molding process such that turbulence of a vascular blood flow is minimized during use. A single molded configuration may be machined or molded and has no seams or parts than require adhesive or fastening, for example.

The lid 400 may include one or more fastener receiver apertures 410 which correspond to the one or more fastening apertures 313 described in FIG. 3A. Although two fastener receiver apertures 410 are shown, one fastener receiver aperture 410, two fastener receiver apertures 410, fastener receiver apertures 410, etc., may be used. Fastener receiver apertures 410 may be configured to receive a fastener for fastening the housing 302 of the port device 300 to the lid 400. The fastener may by any type known in the art including a screw, a bolt, a nail, etc. In other embodiments, the housing 302 and the lid 400 may be fastened using other mechanisms including an adhesive, magnetization, forming the housing 302 and the lid 400 as an integral piece, etc.

FIG. 4B is a top view of the lid of FIG. 4A according to an embodiment of the present invention. As shown in FIG. 4B, an alternating arrangement of ridge elements 406 and groove elements 408 where each element terminates adjacent to an outer perimeter 322 of the tapered seat. FIG. 4B illustrates a configuration where a top of each ridge element 406 is flat and a bottom of each groove element 408 is flat. In other embodiments, where a top of each ridge element 406 is rounded and a bottom of each groove element 408 is rounded. In yet other embodiments, a combination of flat and rounded ridge elements 406 and/or groove elements 408 may be present.

FIG. 4C is a side view of the lid of FIG. 4A according to an embodiment of the present invention. As illustrated in FIG. 4A, the side edges of the lid 400 may be rounded (e.g., no texture or fluted features) although other configurations may be possible. FIG. 4A includes a nozzle piece 420 which is insertable into the inlet opening 304 of the port device 300. In various embodiments, a compliant material (such as an O-ring) may encircle the nozzle piece 420 and further facilitate coupling of the lid 400 to the port device 300 and provide sealing therebetween.

FIG. 4D is a cross-sectional side view of the lid of FIG. 4A according to an embodiment of the present invention. In this view, the fastening receiver apertures 410 are removed for clarity although it would be appreciated by one having ordinary skill in the art that the fastening receiver apertures 410 would extend through at least a portion of the height of the lid 400. FIG. 4D illustrates a second conical surface 422 from which each ridge element 406 and/or each groove element 408 (numbering removed for simplicity) extends or depresses from, respectively. The angle of the second conical surface 422 is within a range from 10 degrees and 25 degrees, inclusive, relative to an otherwise substantially flat top surface of the guide 402. The second conical surface 422 terminates and provides access to the inlet opening 304 of the port device 300.

FIG. 4E is a bottom view of the lid of FIG. 4A according to an embodiment of the present invention. In this view, the fastening receiver apertures 410 are shown and the nozzle piece 420 which is insertable into the inlet opening 304 of the port device 300. In various embodiments, a compliant material (such as an O-ring) may encircle the nozzle piece 420 and further facilitate coupling of the bottom surface 424 of the lid to the top surface 308 of the port device 300 and provide sealing therebetween.

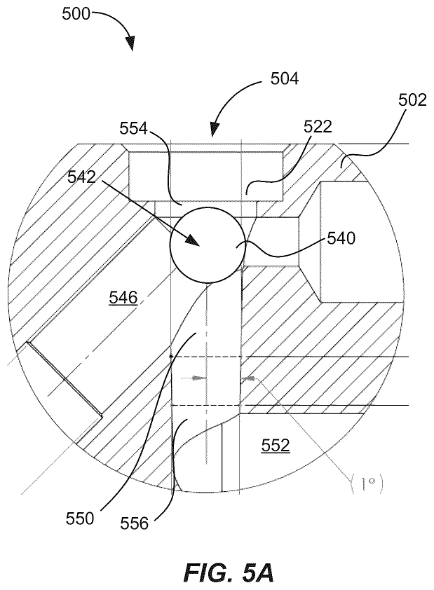

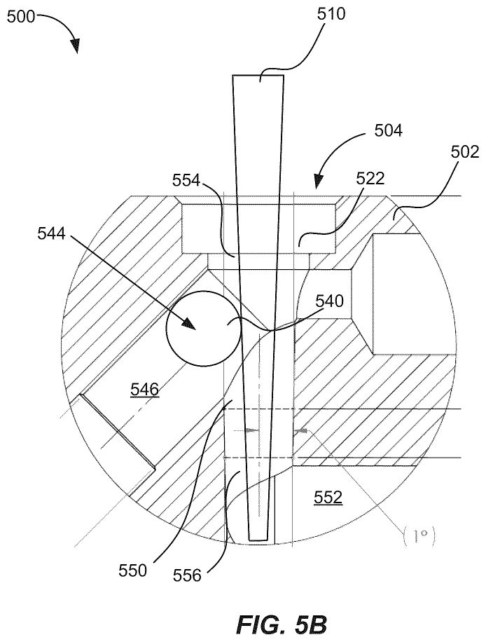

FIG. 5A is a cross-sectional view of a valve mechanism in a first position according to an embodiment of the present invention. As illustrated in FIG. 5A, a valve mechanism 540 is disposed in the housing 502 at a first position 542 proximal to the outer perimeter 522 of the tapered seat 550 at the inlet opening 504. As shown, the valve mechanism 540 includes a spherical element. The inlet opening 504 and/or the proximal portion 554 of the tapered seat 550 are closed to blood flow when the interior conduit 552 and the distal portion 556 of the tapered seat 550 are occluded by the valve mechanism 540 (e.g., a moveable spherical element (ball)). The spherical element may include synthetic sapphire, stainless steel, or other hard material that is hard and lubricious. When an access tube is inserted through the inlet opening 504, the valve mechanism 540 is laterally displaced by the access tube as the access tube is displaced by the access tube.

The valve mechanism 540 is configured to seal the interior conduit 552 closed to physiologic pressures while allowing for vascular blood flow in the first position 542. The valve mechanism 540 is configured to be displaced to a second position 544 via percutaneous insertion of an access tube into the tapered seat 550 to open the valve mechanism 540 and allow continued vascular blood flow through the interior conduit 552. According to at least some embodiments, the second position 544 includes the valve mechanism 540 being at least partially disposed in a valve conduit 546. The valve conduit 546 extends as part of the valve mechanism 540 that extends 45 degrees from a longitudinal axis of the housing 502. The valve conduit 546 provides a space for the movement of the valve mechanism 540. In some embodiments, valve conduit 546 may include an elastomeric spring or silicone ring (both not shown), to accommodate the displaced valve mechanism 540. Other material that adds a resistant force against a displaced valve mechanism 540 may be used in the valve conduit 546.

FIG. 5B is a cross-sectional view of a valve mechanism in a second position according to an embodiment of the present invention. The valve mechanism 540 is configured to be displaced to a second position 544 via percutaneous insertion of an access tube 510 into the tapered seat 550 to open the valve mechanism 540 and allow continued vascular blood flow through the interior conduit 552. According to at least some embodiments, the second position 544 includes the valve mechanism 540 being at least partially disposed in a valve conduit 546. The valve conduit 546 extends as part of the valve mechanism 540 that extends 45 degrees from a longitudinal axis of the housing 502. The valve conduit 546 provides a space for the movement of the valve mechanism 540. In some embodiments, valve conduit 546 may include an elastomeric spring or silicone ring (both not shown), to accommodate the displaced valve mechanism 540. Other material that adds a resistant force against a displaced valve mechanism 540 may be used in the valve conduit 546.

The valve mechanism 540 in the second position 544 allows for continued vascular blood flow through the interior conduit 552 unobstructed by the access tube. The valve mechanism 540 is configured to return to the first position 542 and resume sealing of the interior conduit 552 upon removal of the access tube 510 from the tapered seat 550 at a conclusion of a treatment session or upon removal of the access tube 510 from the tapered seat 550 for any other reason.

In various embodiments, the valve mechanism 540 is configured to be displaced from the first position 542 to the second position 544 by application of an actuation pressure that is significantly less than conventional needle-actuated valve mechanisms (e.g., via insertion of the access tube 510). Advantageously, the design of the valve mechanism 540 described according to embodiments included herein requires significantly less pressure to actuate than traditional valve mechanisms. A patient experiences significantly less discomfort when less pressure is applied to a port device (e.g., when access to vascular blood flow is established). For example, having the valve mechanism 540 more proximate to the inlet opening 504 compared to other designs increases the mechanical advantage at the beginning of the percutaneous insertion of the access tube 510 into the port device 500. Less pressure on the valve mechanism 540 results in less pain to the patient as the valve mechanism 540 is displaced and reduces injury to the tissue pocket supporting the implanted device.

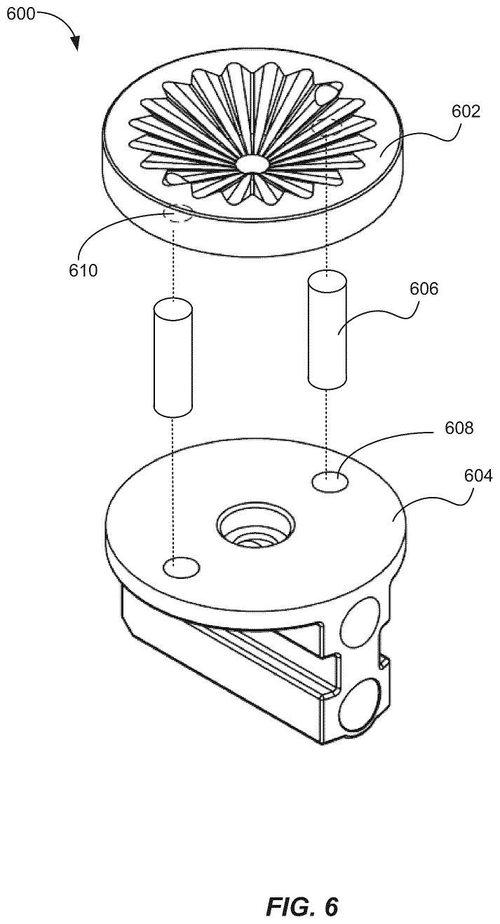

FIG. 6 is an exploded view of a two-part implantable graft system according to an embodiment of the present invention. The two-part implantable graft system 600 includes a lid 602 (such as lid 400 described with respect to FIGS. 4A-4E), a port device 604 (such as port device 300 described with respect to FIGS. 3A-3H), and one or more fasteners 606. In various embodiments, the one or more fasteners 606 may be any type known in the art and are configured to mate with corresponding aperture features 608 of the port device 604 and aperture receiving features 610 of the lid 602. It should be understood by one having ordinary skill in the art that the lid 602 and the port device 604 may be fastened by other means including, but not limited to, adhesives, magnets, electrical mechanical means, etc. In yet other embodiments, the lid 602 and the port device 604 may be an integrally formed piece.

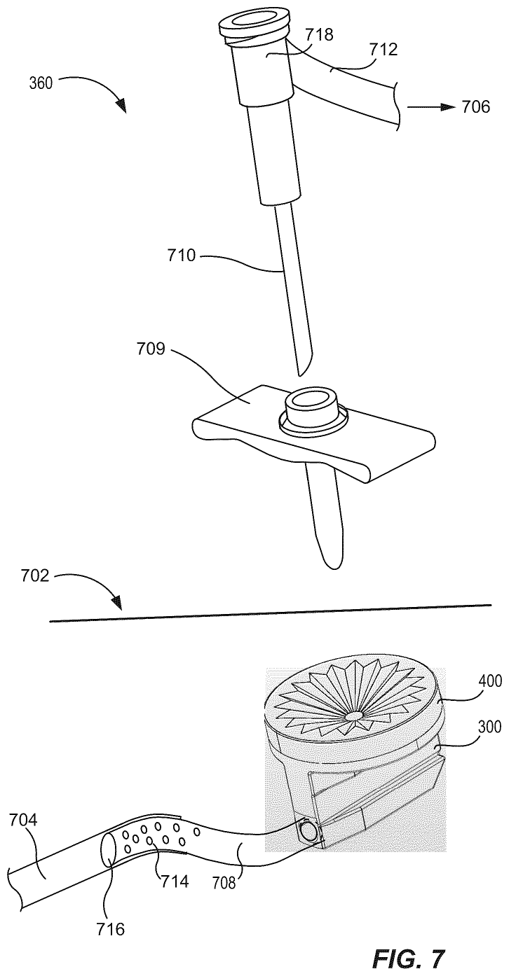

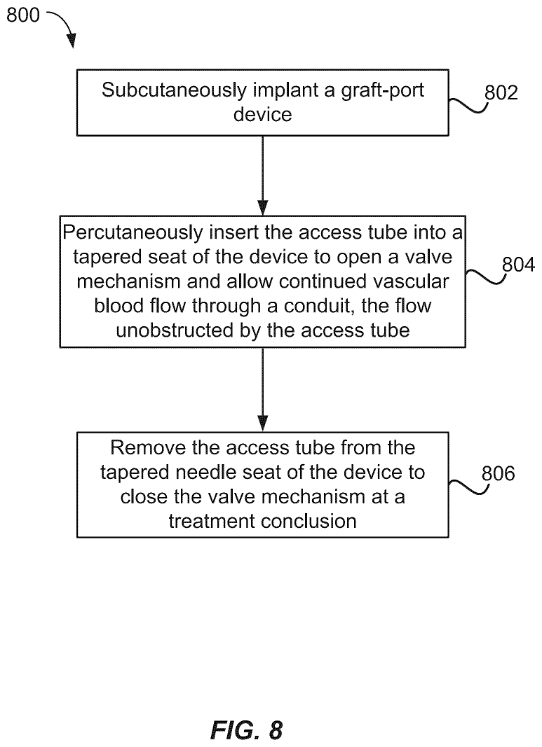

FIG. 7 a view of an assembly including a two-part implantable graft system according to an embodiment of the present invention. The two-part implantable graft system comprises the port device 300 and the lid 400 as described in detail above. The port device 300 and the lid 400 are implanted under the skin 702 between a target blood vessel 704, typically a vein and the skin 702. During hemodialysis, blood 706 may be withdrawn through a catheter 708, through the port device 300 and the lid 400, and externally through an access tube 710 and or a trocar 709 that are inserted through the lid 400 and the port device 300 (such as through the inlet opening 304 described in detail above), and through a connecting line 712 used to percutaneously access the two-part implantable graft system. Alternatively, the two-part implantable graft system and the catheter could be used to return treated blood to the patient.

In various embodiments, the access tube 710 may include a swivel mechanism 718 to attach the connecting line 712. The swivel mechanism 718 is capable of rotating 360 degrees in a clockwise or counterclockwise direction to prevent the connecting line 712 from kinking or binding if the patient moves or otherwise changes position. In addition to allowing patient mobility, the swivel also prevents accidental decannulation.

In some embodiments, the catheter 708 may contain pores 714 and/or a central opening 716. Central opening 716 interfaces with an opening of the port device 300 (such as outlet opening 306 as described in detail above). The catheter 708 interfaces with the blood vessel 704 or other body space filled with (or potentially filled with) a fluid (i.e., blood, urine, cerebral-spinal fluid). It is contemplated that the two-part implantable graft system can be used for various therapies beyond hemodialysis with little or no modification. Some other therapeutic uses may include peritoneal dialysis, urinary tract drainage, pleural effusion, and cerebral-spinal fluid drainage. The two-part implantable graft system may be used to administer insulin or even inflate a penile prosthetic, for example.

FIG. 8 is a flowchart of a method according to an embodiment of the present invention. Method 800 includes step 802. Step 802 includes subcutaneously implanting a graft-port device (such as a two-part graft-port system described throughout the present disclosure including a includes a lid (such as lid 400 described with respect to FIGS. 4A-4E), a port device (such as port device 300 described with respect to FIGS. 3A-3H)). Step 804 includes percutaneously inserting the access tube into a tapered seat of the device to open a valve mechanism and allow continued vascular blood flow through a conduit, the flow unobstructed by the access tube. Step 804 may include displacing the valve mechanism from a first position to a second position, as described in detail above, especially with reference to FIGS. 5A-5B. Step 806 includes removing the access tube from the tapered needle seat of the device to close the valve mechanism at a treatment conclusion. As described above, removing the access tube from the tapered needle seat of the device enables the valve mechanism to return from the second position to the first position, thereby resuming sealing of the tapered seat from the vascular blood flow.

Additional embodiments of devices, methods, and systems related to a subcutaneously implanted port device as described herein are included in U.S. Pat. No. 10,456,570 entitled “Graft-Port Hemodialysis Systems, Devices, and Methods,” the entire disclosure of which is incorporated herein by reference.

According to various methods known in the art, a catheter may be coupled to a port system via a barbed port stem inserted into the catheter lumen. A barb includes projections that, while securing the port stem into the catheter, also form a potential micro abscess space that is susceptible to infection or the like. Various embodiments described herein address the foregoing deficiency by providing a connector device that couples the catheter to the port system, and particularly couples the catheter to the port stem.

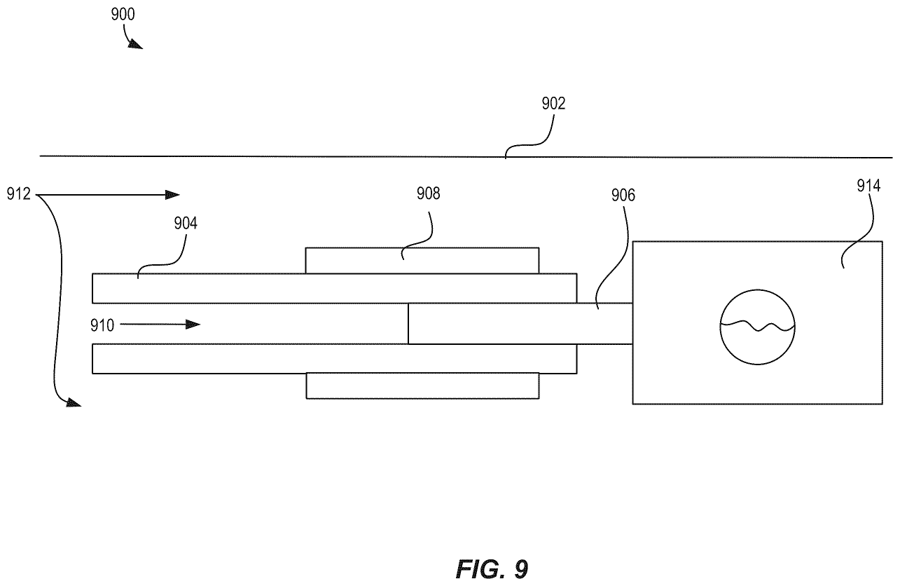

FIG. 9 illustrates an exemplary connector device for a catheter of an implantable graft system. The implantable graft system 900 may be located under a skin surface 902 of a patient according to any of the embodiments described in detail above. Furthermore, the implantable graft system 900 may incorporate any embodiment described in the present disclosure as would be appreciated by one having ordinary skill in the art.

As illustrated in FIG. 9, a catheter 904 is coupled to a port stem 906 via a connector device 908. The connector device 908 may grip and secure the catheter 904 in position relative to the port stem 906. In various embodiments, the connector device 908 is porous. Furthermore, the connector device 908 reticulated or trabecular according to exemplary embodiments. The reticulated or trabecular nature of the connector device 908 may be based at least in part on the material forming the connector device 908. For example, the connector device 908 may include silicon nitride. In at least some embodiments, the connector device 908 is formed from silicon nitride. Silicon nitride provides radiopacity for the connector device 908, as would be appreciated by one having ordinary skill in the art in view of the present disclosure. The connector device 908 may be formed via additive manufacturing methods known in the art and/or other formation methods as would be appreciated by one having ordinary skill in the art in view of the present disclosure. For example, the connector device 908 may be formed by 3D printing techniques or via injection molding.

In various embodiments, the connector device 908 provides a firm pressure to grip the catheter 904 to the port stem 906. The connector device 908 may be formed of an infection resistant material (e.g., silicon nitride) with a reticulated or trabecular structures to encourage tissue ingrowth. Advantageously, the reticulated or trabecular configuration of the connector device 908 enables tissue ingrowth that secures the catheter in position and further eliminates any space for abscess formation between the catheter 904 and the port stem 906. For example, the connector device 908 seals and isolates the connection between the catheter 904 and the port stem 906. Furthermore, the connector device 908 secures the transcutaneous catheter (e.g., catheter 904) in its subcutaneous tunnel 912 by enabling tissue ingrowth and preventing accidental removal.

According to at least some embodiments, the catheter 904, the port stem 906, the connector device 908, etc., or any combination thereof, may be microporous or nanoporous such that components of the implantable graft system 900 may be impregnated with a lock solution or the like. According to some embodiments, various components may include PEEK, PEKK, etc. In at least some embodiments, the porosity of one or more of the catheter 904, the port stem 906, or the connector device 908 may be tuned as desired. For example, the porosity of the port stem 906 may be tuned using sodium chloride. In various embodiments, the porosity of one or more of the catheter 904, the port stem 906, or the connector device 908 may be tuned to the molecular size of a lock solution used with the implantable graft system 900.

In at least some embodiments, an antimicrobial, anticoagulant, antiseptic, etc. lock solution may be applied within a catheter lumen 910 of the implantable graft system 900. In other embodiments, an antimicrobial, anticoagulant, antiseptic, etc., reservoir 912 may be provided in the implantable graft system 900 and coupled to the port stem 906. Various embodiments of the present disclosure create an antimicrobial, anticoagulant, antiseptic, etc., catheter connection from the catheter 904 to the port stem 906. An antimicrobial, anticoagulant, antiseptic, etc., lock solution may include at least 10% sodium chloride and at least 3% acetic acid, according to at least some embodiments. In exemplary embodiments, the lock solution may include 3% to 23.5% sodium chloride and 3% to 10% acetic acid. In some embodiments, the lock solution may include hypertonic sodium chloride and 3% to 10% acetic acid.

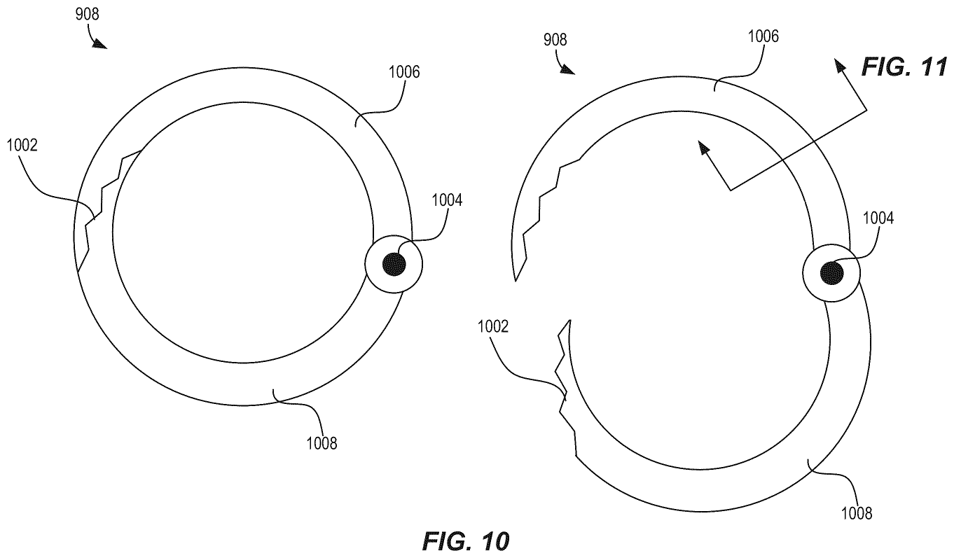

FIG. 10 is a view of the connector device of FIG. 9 in an open position and a closed position. The connector device 908 may include a closing mechanism 1002. As shown in FIG. 10, the closing mechanism 1002 includes a ratchet closure, although any closing mechanism may be used for the connector device 904. In exemplary embodiments, the closing mechanism 1002 does not create a space for an infection to form (e.g., the ratchet closure creates a seamless seal when in the closed position). In various embodiments, the connector device 908 may further include a hinge 1004 for coupling a first portion 1006 and a second portion 1008 of the connector device 908 relative to one another from the open position to the closed position, and vice versa. In various embodiments, the connector device 908 may be referred to as a ‘clamshell’ configuration. In other embodiments, the connector device 908 may include more than two portions and one hinge 1004 as shown in FIG. 10.

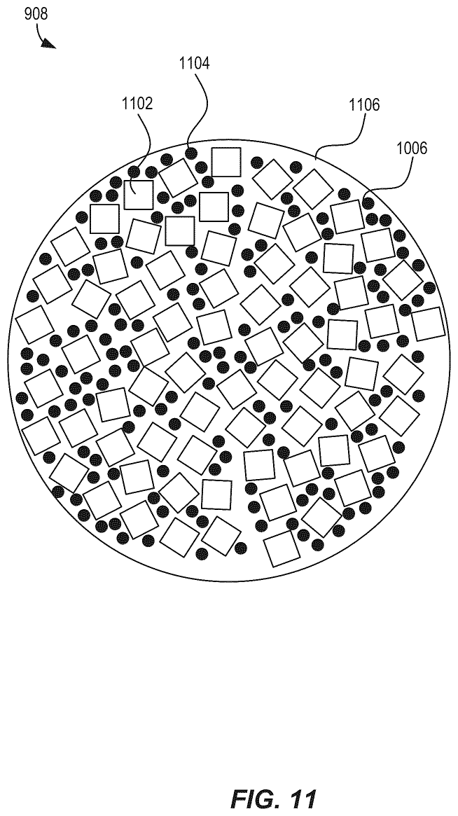

FIG. 11 is a cross-sectional view of the connector device of FIG. 9. Each of the first portion 1006 and the second portion 1008 may include the same cross-section as shown in FIG. 11. As described with respect to FIG. 9, the connector device 908 may include silicon nitride. In some embodiments, the connector device 908 is formed of sodium chloride 1102, having a natural cubic crystalline, and a fine silicon nitride particulate 1104. Together the sodium chloride 1102 and the silicon nitride particulate 1104 create an “open cell” wall. In some embodiments, the surface of the silicon nitride particulate 1104 is exposed as the sodium chloride 1102 dissolves. For example, the silicon nitride particulate 1104 may remain in the embedded in a polymer 1106 or the like.

Although embodiments of the invention have been described in considerable detail with reference to certain preferred versions thereof, other embodiments are possible. Therefore, the spirit and scope of the appended claims should not be limited to the descriptions of the embodiments above.

1. A subcutaneously implanted graft-port device used to establish access to a blood vessel of a patient, the patient requiring repeated vascular access over a period of time, the device comprising:
a housing having an inlet opening, an outlet opening, and an interior conduit defined therebetween, the interior conduit configured to accept a vascular blood flow, wherein the housing includes a substantially flat surface oriented proximal to and substantially parallel with a skin of the patient when the device is subcutaneously implanted;
a tapered seat disposed in a center of the housing, wherein the tapered seat includes an outer perimeter, an inner perimeter smaller than the outer perimeter, the tapered seat configured to receive an access tube through the outer perimeter at the inlet opening; and
a valve mechanism disposed in the housing at a first position proximal to the outer perimeter of the tapered seat at the inlet opening, the valve mechanism configured to seal the interior conduit closed to physiologic pressures while allowing for vascular blood flow in the first position, wherein the valve mechanism is configured to be displaced to a second position via percutaneous insertion of the access tube into the tapered seat to open the valve mechanism and allow continued vascular blood flow through the interior conduit.

2. The device of claim 1, further comprising a sealing element located at the outer perimeter of the tapered seat configured to seal the valve mechanism, wherein the sealing element is an O-ring.

3. (canceled)

4. The device of claim 1, wherein the valve mechanism is configured to return to the first position and resume sealing of the interior conduit upon removal of the access tube from the tapered seat at a conclusion of a treatment session, wherein the treatment session is selected from the group consisting of hemodialysis, hemofiltration, hemodiafiltration, plasmapheresis, ultrafiltration, aquapheresis, lipid pheresis, chemotherapy, hemoperfusion, peritoneal dialysis, and pleural drainage.

5. The device of claim 1, wherein the valve mechanism extends 45 degrees from a longitudinal axis of the housing.

6. The device of claim 1, wherein the tapered seat comprises a conical surface extending between the outer perimeter and the inner perimeter, wherein the tapered seat comprises a taper angle of at least or less than 1 degree from the outer perimeter to the inner perimeter.

7. (canceled)

8. The device of claim 1, wherein the valve mechanism in the second position allows for continued vascular blood flow through the interior conduit unobstructed by the access tube.

9. The device of claim 1, wherein the valve mechanism is a movable spherical element.

10. (canceled)

11. The device of claim 1, further comprising a lid configured to couple with the substantially flat surface of the housing, wherein the lid includes a continuous sequence of fluted features.

12. (canceled)

13. The device of claim 11, wherein the fluted features include at least one ridge element elevated from a surface, wherein each ridge element terminates adjacent the outer perimeter of the tapered seat.

14. The device of claim 13, wherein the fluted features include at least one groove element depressed from the surface, wherein each groove element terminates adjacent the outer perimeter of the tapered seat, and wherein the at least one groove element alternates with the at least one ridge element.

15. The device of claim 11, wherein the fluted features are configured and located to guide a tip of the access tube toward the outer perimeter of the tapered seat so as to facilitate percutaneous introduction of the access tube into the tapered seat, wherein the access tube is a needle.

16. The device of claim 1, wherein the housing further includes a transparent material, one or more light emitting diodes, and a receiver coil for receiving an electrical current, wherein the one or more light emitting diodes are configured to illuminate the tapered seat when an electromagnetic induction chip is placed external to the patient and substantially above the subcutaneously implanted device, the electromagnetic induction chip producing a voltage in the receiver coil, the voltage powering the one or more light emitting diodes so that the tapered seat of the subcutaneously implanted device is visible to a user of the device.

17. (canceled)

18. The device of claim 1, wherein at least a portion of the housing is sufficiently nano-porous so as to permit tuned diffusion of an anti-microbial agent outward from the interior conduit of the housing to an outer surface of the housing so that infection is inhibited.

19. (canceled)

20. The device of claim 1, further comprising a lock solution comprises 3% to 23.5% sodium chloride and 3% to 10% acetic acid.

21.-33. (canceled)

34. two-part implantable port system for establishing access to a blood vessel of a patient, the system comprising:
a port device, the port device comprising:
a substantially flat surface, the substantially flat surface oriented proximal to and substantially parallel with a skin of the patient when the device is subcutaneously implanted;
a tapered seat disposed in a center of the substantially flat surface and configured to receive a tip of an access tube, the tapered seat having a proximal portion, a distal portion, and a first conical surface extending between the proximal portion and the distal portion, wherein the proximal portion of the tapered seat is configured to receive the tip of the access tube therethrough; and
an interface surface configured to engage (i) the blood vessel of the patient or (ii) a vascular access catheter, the interface surface having an aperture in fluid communication with the distal portion of the tapered seat; and
a lid configured to couple to the substantially flat surface of the port device, the lid comprising:
a guide configured to engage the tip of the access tube and to facilitate percutaneous introduction of the access tube toward the tapered seat irrespective of an introduction angle, wherein the guide includes a continuous sequence of fluted features.

35.-42. (canceled)

43. An implantable port device for establishing access to a blood vessel of a patient, the port device comprising:
a substantially flat surface, the substantially flat surface oriented proximal to and substantially parallel with a skin of the patient when the port device is subcutaneously implanted;
a tapered seat disposed in a center of the substantially flat surface and configured to receive a tip of an access tube, the tapered seat having a proximal portion, a distal portion, and a conical surface extending between the proximal portion and the distal portion, wherein the proximal portion of the tapered seat is configured to receive the tip of the access tube therethrough;
a port stem having an aperture in fluid communication with the distal portion of the tapered seat;
a vascular access catheter configured to engage the port stem; and
a connector device for coupling the vascular access catheter to the port stem;
wherein at least a portion of the connector device is sufficiently nano-porous so as to permit tuned diffusion of an anti-microbial agent outward from a conduit of the port device to an outer surface of the port device so that infection is inhibited.

44. The device of claim 43, wherein the connector device promotes tissue ingrowth under the skin of the patient, wherein the connector device secures the vascular access catheter in a subcutaneous tunnel via the tissue ingrowth.

45. (canceled)

46. The device of claim 43, wherein the connector device comprises silicon nitride.

47. The device of claim 43, wherein the connector device comprises a closure mechanism for assuring pressure on the vascular access catheter, wherein the closure mechanism comprises a hinge and the connector device comprises a clamshell configuration.

48. (canceled)

49. The device of claim 43, wherein the vascular access catheter is microporous.
